(19) United States
(12) Patent Application Publication
(10) Pub. No.: US 2026/0358153 A1
(43) Pub. Date: Nov. 12, 2026

(54) ERROR DETECTION METHOD OF PAVEMENT WEAR DETECTION DEVICE

(71) Applicant: Research Institute of Highway Ministry of Transportation, Beijing (CN)

(72) Inventors: Jinning ZHANG, Beijing (CN); Yue Liu, Beijing (CN); Yishu Zhou, Beijing (CN); Hongbo Guo, Beijing (CN); Lishuo Ren, Beijing (CN); Jinjin Cao, Beijing (CN)

(21) Appl. No.: 19/442,424

(22) Filed: Jan. 7, 2026

(30) Foreign Application Priority Data
Jan. 17, 2025 (CN) 202510072314.0

(51) Int. Cl. G01D 18/00; G01B 11/30; G01N 33/42

(52) U.S. Cl. CPC G01D 18/00; G01B 11/303; G01B 11/306; G01N 33/42

(57) ABSTRACT

The present disclosure relates to the field of pavement detection, in particular to an error detection method of a pavement wear detection device, and the error detection method includes the following steps: acquiring pavement morphology information; performing fitting processing on the pavement morphology information by using a preset data fitting processing algorithm to obtain at least one texture depth variation curve; determining a first pavement wear value based on each texture depth variation curve and preset configuration information; generating test plates in one-to-one correspondence with each texture depth variation curve based on each texture depth variation curve; detecting the test plate with the pavement wear detection device to determine a second pavement wear value; and determining a detection error of the pavement wear detection device based on the first pavement wear value and the second pavement wear value.

### CROSS-REFERENCE TO RELATED APPLICATION

This application claims priority to China Patent Application 202510072314.0, filed Jan. 17, 2025, which is incorporated herein by reference.

### TECHNICAL FIELD

The present disclosure relates to the field of pavement detection, and in particular to an error detection method of a pavement wear detection device.

### BACKGROUND

In recent years, with the rapid development of detection technology and the continuous iteration of detection device, detection device with multiple pavement detection functions has been widely used in the field of road condition assessment. The detection device is based on multi-point laser technology, image processing technology or positioning technology, and detection results that characterize the degree of pavement wear can be obtained by driving on the pavement with assessment requirements.

However, the use of relevant technologies for pavement wear detection will lead to detection errors in the pavement wear detection device due to complex and changeable road conditions, aging of detection device, and interference from the external environment. Since the detection error cannot be accurately quantified based on the detection results of the detection device, it is difficult to calibrate, analyze and correct the detection error of the pavement wear detection device.

### SUMMARY

In order to overcome the problems existing in the related art, the present application provides an error detection method of a pavement wear detection device.

According to some embodiments of the present application, an error detection method of pavement wear detection device is provided, includes:
acquiring pavement morphology information, the pavement morphology information is used to characterize elevation changes of a plurality of original pavements;
performing fitting processing on the pavement morphology information by using a preset data fitting processing algorithm to obtain at least one texture depth variation curve;
determining a first pavement wear value based on each texture depth variation curve and preset configuration information, wherein the preset configuration information is used to characterize a corresponding relationship between a texture depth and a pavement wear value;
generating a test plate in one-to-one correspondence with each texture depth variation curve based on each texture depth variation curve, wherein the extension path of the edge of the test plate is consistent with the corresponding texture depth variation curve;
detecting the test plate with the pavement wear detection device, so that a second pavement wear value is determined based on the test plate and the preset configuration information with the pavement wear detection device; and
determining a detection error of the pavement wear detection device based on the first pavement wear value and the second pavement wear value.

In some embodiments of the present application, the pavement morphology information includes a plurality of elevation values corresponding to each of the original pavements respectively;
the performing fitting processing on the pavement morphology information by using a preset data fitting processing algorithm to obtain at least one texture depth variation curve includes:
dividing the plurality of elevation values into a plurality of first initial elevation value sets based on the regional location information of each of the original pavements, wherein the plurality of first initial elevation value sets corresponds one-to-one to a plurality of preset geographical areas;
determining the first initial elevation value set corresponding to the target geographical area as a first target elevation value set, and performing fitting processing on the elevation values in the first target elevation value set to obtain at least one texture depth variation curve; or,
dividing the plurality of elevation values into a plurality of second initial elevation value sets based on the preset elevation value range in which each elevation value is located, wherein the plurality of second initial elevation value sets corresponds one-to-one to a plurality of preset texture depth ranges;
taking the second initial elevation value set having the largest number of elevation values as a second target elevation value set, and performing fitting processing on the elevation values in the second target elevation value set to obtain a fitting curve, wherein the fitting curve matches the preset texture depth range corresponding to the second target elevation value set;
determining at least one mapping curve based on the fitting curve, ensures that each mapping curve matches the preset texture depth range corresponding to any one of the second initial elevation value sets other than the second target elevation value set;
taking the fitting curve and each mapping curve as a plurality of the texture depth variation curves.

In some embodiments of the present application, the fitting process includes any one or any combination of linear regression fitting processing, polynomial fitting processing, spline interpolation fitting processing, surface fitting processing, and least squares fitting processing.

In some embodiments of the present application, the texture depth includes a left wheel track band texture depth, a lane center line texture depth, and a right wheel track band texture depth;
the number of the texture depth variation curve is greater than or equal to 3.

In some embodiments of the present application, wherein the determining a first pavement wear value based on each texture depth variation curve and preset configuration information includes:
determining any three of the texture depth variation curves as three target texture depth variation curves;
determining the first left wheel track band texture depth, the first lane center line texture depth and the first right wheel track band texture depth of the first preset position based on the three target texture depth variation curves respectively;
determining the first pavement wear value based on the first left wheel track band texture depth, the first lane center line texture depth, the first right wheel track band texture depth and the preset configuration information;
the detecting the test plate with the pavement wear detection device, so that a second pavement wear value is determined based on the test plate and the preset configuration information with the pavement wear detection device includes:
detecting the three test plates generated respectively based on the three target texture depth variation curves with the pavement wear detection device, ensures that the second left wheel track band texture depth, the second lane center line texture depth and the second right wheel track band texture depth at the second preset position are determined based on the three test plates respectively with the pavement wear detection device, and the second pavement wear value is determined based on the second left wheel track band texture depth, the second lane center line texture depth, the second right wheel track band texture depth and the preset configuration information; wherein the first preset position of the target texture depth variation curve matches the second preset position of the test plate.

In some embodiments of the present application, different target texture depth variation curves match different preset texture depth ranges;
among the maximum values of the three preset texture depth ranges respectively matched by the three target texture depth variation curves, the maximum value of the preset texture depth range matched with the target texture depth variation curve used to determine the first lane center line texture depth is the smallest.

In some embodiments of the present application, after generating the test plate in one-to-one correspondence with each texture depth variation curve based on each texture depth variation curve, the error detection method of the pavement wear detection device further includes:
performing a process error detection on each generated test plate to determine the dimensional error between the extension path of the edge of the test plate and the texture depth variation curve caused by the processing technology of the test plate;
the detecting the test plate with the pavement wear detection device includes:
detecting the test plate whose dimensional error is less than a preset error with the pavement wear detection device.

In some embodiments of the present application, the generation method of the test plate includes any one or a combination of three-dimensional printing, precision machining, and three-axis computer numerical control machining.

In some embodiments of the present application, the material of the test plate is aluminum, the thickness of the test plate is 0.5-1.5 cm, and the surface of the test plate is matte treated.

In some embodiments of the present application, the error detection method of the pavement wear detection device further includes:
based on the detection error, performing any one or any combination of the following processes to the pavement wear detection device:
detection error calibration processing;
analysis and processing of the cause of detection error;
detection parameter correction processing.

The technical solution provided by the embodiment of the present application may include the following beneficial effects: by generating the texture depth variation curve and the test plate corresponding to the texture depth variation curve, the first pavement wear value under a standard theoretical calculation condition can be determined according to the texture depth variation curve, and the second pavement wear value under an actual detection condition can be obtained according to the test plate and the pavement wear detection device, thereby determining the detection error of the pavement wear detection device according to the difference between the first pavement wear value and the second pavement wear value, realizing the accurate quantification of the detection error of the pavement wear detection device, and providing a basis for the calibration, analysis and correction of the detection error. A large amount of raw data can be fitted by a preset data fitting processing algorithm, so that the texture depth variation curve can meet the needs of different scenarios to characterize complex and diverse road conditions. By generating the test plate, the road conditions represented by the texture depth variation curve are truly restored, so that the actual detection result corresponding to the standard theoretical calculation result can be obtained by detecting the test plate with the pavement wear detection device, thereby ensuring the authenticity of the detection error and improving the applicability and accuracy of the error detection method.

It should be understood that the foregoing general description and the following detailed description are exemplary and explanatory only and are not restrictive of the present application.

### BRIEF DESCRIPTION OF THE DRAWINGS

The accompanying drawings incorporated into the specification and constituting a part of the specification illustrate the embodiments of the present disclosure, and are used together with the description to explain the principles of the embodiments of the present disclosure.

FIG. 1 is a flowchart of the error detection method of a pavement wear detection device according to an exemplary embodiment.

FIG. 2 is a schematic diagram of the test plate according to an exemplary embodiment.

FIG. 3 is a flowchart of performing fitting processing on pavement morphology information using a preset data fitting processing algorithm to obtain at least one texture depth variation curve according to an exemplary embodiment.

FIG. 4 is a flowchart showing a method of fitting pavement morphology information with a preset data fitting processing algorithm to obtain at least one texture depth variation curve according to another exemplary embodiment.

FIG. 5 is a flowchart of determining the first pavement wear value based on each texture depth variation curve and the preset configuration information according to an exemplary embodiment.

### DETAILED DESCRIPTION

Exemplary embodiments will be described in detail herein, examples of which are shown in the accompanying drawings. When the following description refers to the drawings, the same numbers in different drawings represent the same or similar elements unless otherwise indicated. The embodiments described in the following exemplary embodiments do not represent all embodiments consistent with the present disclosure. Instead, they are merely examples of devices and methods consistent with some aspects of the present disclosure as detailed in the accompanying claims.

In recent years, with the rapid development of road condition detection technology and the continuous iteration of detection device, detection device with multiple pavement detection functions, such as multi-functional detection vehicles, has been widely used in the field of road condition assessment. The detection device is based on detection technologies such as multi-point laser technology, image processing technology or positioning technology. By driving on the pavement with evaluation requirements, the detection results representing the degree of pavement wear can be obtained to determine the wear condition of the pavement structure.

However, due to the complex and changeable road conditions of the target road, the aging of the detection device, and interference from the external environment, the pavement wear detection device has detection errors when conducting pavement wear detection. Since it is impossible to accurately quantify the detection error based solely on the detection results of the detection device, it is difficult to calibrate, analyze and correct the detection error of the pavement wear detection device, which seriously affects the use effect and experience of the pavement wear detection device.

Based on this, the exemplary embodiment of the present application provides an error detection method for a pavement wear detection device, by generating the texture depth variation curve and the test plate corresponding to the texture depth variation curve, the first pavement wear value under a standard theoretical calculation condition can be determined according to the texture depth variation curve, and the second pavement wear value under an actual detection condition can be obtained according to the test plate and the pavement wear detection device, thereby determining the detection error of the pavement wear detection device according to the difference between the first pavement wear value and the second pavement wear value, realizing the accurate quantification of the detection error of the pavement wear detection device, and providing a basis for the calibration, analysis and correction of the detection error. A large amount of raw data can be fitted by a preset data fitting processing algorithm, so that the texture depth variation curve can meet the needs of different scenarios to characterize complex and diverse road conditions. By generating the test plate, the road conditions represented by the texture depth variation curve are truly restored, so that the actual detection result corresponding to the standard theoretical calculation result can be obtained by detecting the test plate with the pavement wear detection device, thereby ensuring the authenticity of the detection error and improving the applicability and accuracy of the error detection method.

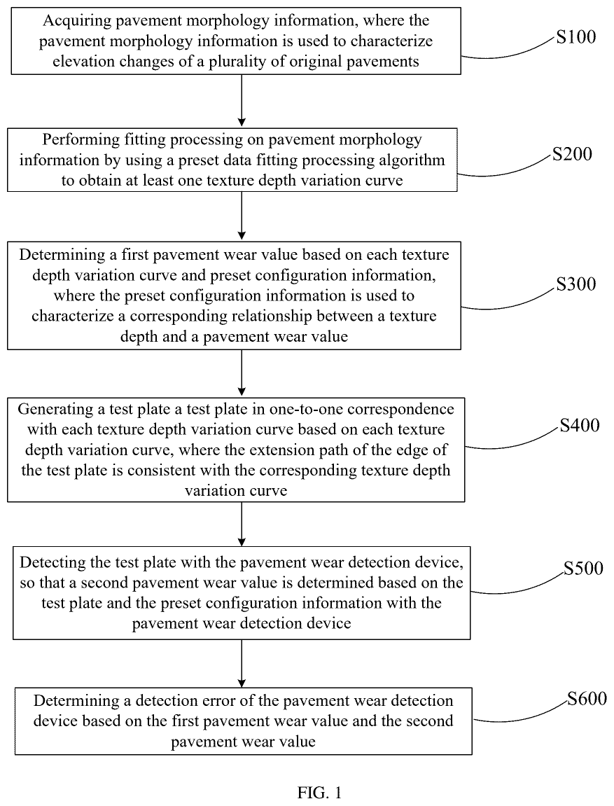

In an exemplary embodiment, an error detection method of a pavement wear detection device is provided, referring to FIG. 1 as shown, the error detection method of the pavement wear detection device includes:

S100: Acquiring pavement morphology information, where the pavement morphology information is used to characterize elevation changes of a plurality of original pavements.

In step S100, pavement morphology information is obtained, and the pavement morphology information can characterize the elevation changes of a plurality of original pavements, that is, the changes in the height of the original pavements relative to the reference surface. The pavement morphology information is data obtained by measuring the original pavement, and the pavement morphology information can be, for example, elevation data or point cloud data, etc., which can reflect or extract the pavement elevation.

The location, type and quantity of the original pavements can be selected according to the road condition simulation requirements or error detection requirements. When enough original pavements are selected, the pavement morphology information can include a large amount of data, which can correspond to a variety of different locations and road conditions, making it easy to filter information according to actual needs, so that the error detection method can be applied to different scenarios.

S200: Performing fitting processing on pavement morphology information by using a preset data fitting processing algorithm to obtain at least one texture depth variation curve.

In step S200, fitting processing is a data processing method for fitting a large amount of irregular data into a curve that meets certain characteristics. The preset data fitting processing algorithm is a method for performing the fitting processing. The preset data fitting processing algorithm can be selected according to the fitting requirements or the characteristics of the original data. Exemplarily, the preset data fitting processing algorithm may include classification fitting processing, average fitting processing, threshold fitting processing, etc.

Since the pavement morphology information can characterize the elevation change of the original pavement, it can reflect the texture depth change of the original pavement. The texture depth refers to the depth of the open pores of the uneven pavement, which is an important indicator for measuring the roughness of the pavement and determining the pavement wear. The pavement morphology information is fitted with a preset data fitting processing algorithm to obtain one or more texture depth variation curves, so that each texture depth variation curve can characterize the change of the pavement texture depth, so as to simulate the real road conditions through the numerical curve. The morphology and number of the texture depth variation curves can be selected according to the error detection requirements. Exemplarily, if the pavement wear detection device adopts a three-line detection method to determine the pavement wear value, the number of texture depth variation curves can be set to 3, and the morphology of the three texture depth variation curves are different. Fitting the pavement morphology information with a preset data fitting processing algorithm can fit a large amount of original data, so that the texture depth variation curve can meet the needs of different scenarios and characterize complex and diverse road conditions.

S300: Determining a first pavement wear value based on each texture depth variation curve and preset configuration information, where the preset configuration information is used to characterize a corresponding relationship between a texture depth and a pavement wear value.

In step S300, the preset configuration information can characterize the corresponding relationship between the texture depth and the pavement wear value. The preset configuration information can be, for example, a calculation formula with the texture depth as a variable and the pavement wear value as a calculation result. The preset configuration information can also be other algorithms or models that can determine the pavement wear value according to the texture depth. The pavement wear value can be, for example, the pavement wear index PWI or the wear rate WR proposed in the “Highway Technical Condition Assessment Standard” (JTG5210-2018). In this case, the determination of the pavement wear value requires obtaining three texture depths simultaneously, and at least three texture depth variation curves are required.

The first pavement wear value can be determined according to the texture depth variation curve and the preset configuration information, so that the first pavement wear value is the pavement wear value under the standard theoretical calculation condition corresponding to the texture depth variation curve, i.e., the numerical curve, which represents the detection result that should be obtained if there is no detection error in the pavement wear detection device, and provides a basis for comparison with subsequent actual measurement results.

S400: Generating a test plate in one-to-one correspondence with each texture depth variation curve based on each texture depth variation curve, wherein the extension path of the edge of the test plate is consistent with the corresponding texture depth variation curve.

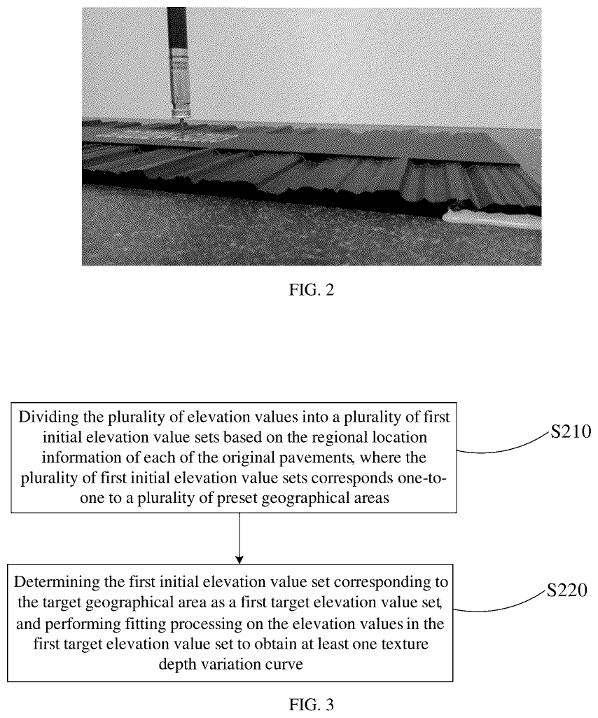

In step S400, for each texture depth variation curve, according to the texture depth variation curve, the corresponding test plate shown in FIG. 2 can be generated through the plate making process, and the extension path of the edge of the test plate is guaranteed to be consistent with the corresponding texture depth variation curve, so that the test plate can truly restore the road conditions represented by the texture depth variation curve, and has the same texture depth as the texture depth variation curve at the same horizontal position. The size and material of the test plate can be selected according to the type of pavement wear detection device and the road conditions to be restored. For example, the length and width of the test plate need to meet the minimum detection range of the pavement wear detection device, and the texture depth variation formed by the edge extension path of the test plate needs to reach the minimum detection accuracy of the pavement wear detection device.

S500: Detecting the test plate with the pavement wear detection device, so that a second pavement wear value is determined based on the test plate and the preset configuration information with the pavement wear detection device.

In step S500, the test plate is detected by the pavement wear detection device. For example, the test plate can be placed on a pavement with a relatively high flatness so as to restore the road condition represented by the texture depth variation curve through the test plate, and the pavement wear detection device is passed through the area where the test plate is located to detect the test plate. After the test plate is detected by the pavement wear detection device, the second pavement wear value can be determined according to the texture depth change of the test plate and the same preset configuration information as when determining the first pavement wear value, so that the second pavement wear value is the pavement wear value when the pavement wear detection device uses the same calculation theory to actually detect the test plate, reflecting the detection effect of the pavement wear detection device on the real pavement.

S600: Determining a detection error of the pavement wear detection device based on the first pavement wear value and the second pavement wear value.

In step S600, since the first pavement wear value and the second pavement wear value are both determined based on the same preset configuration information, i.e., the calculation theory, and the first pavement wear value is the pavement wear value under the standard theoretical calculation corresponding to the texture depth variation curve, and the second pavement wear value is the pavement wear value under the actual detection corresponding to the real pavement restored by the test plate, so that the difference between the first pavement wear value and the second pavement wear value represents the difference between the standard theoretical calculation result and the actual detection result of the pavement wear detection device, and therefore the detection error of the pavement wear detection device can be determined according to the first pavement wear value and the second pavement wear value. Exemplarily, the difference between the first pavement wear value and the second pavement wear value can be used as the detection error of the pavement wear detection device.

In this embodiment, by generating the texture depth variation curve and the test plate corresponding to the texture depth variation curve, the first pavement wear value under the standard theoretical calculation condition can be determined according to the texture depth variation curve, and the second pavement wear value under the actual detection condition can be obtained according to the test plate and the pavement wear detection device, thereby determining the detection error of the pavement wear detection device according to the difference between the first pavement wear value and the second pavement wear value, achieving accurate quantification of the detection error of the pavement wear detection device, and providing a basis for the calibration, analysis and correction of the detection error. A large amount of raw data can be fitted by a preset data fitting processing algorithm, so that the texture depth variation curve can meet the needs of different scenarios to characterize complex and diverse road conditions. By generating the test plate, the road conditions represented by the texture depth variation curve are truly restored, so that the pavement wear detection device can obtain the actual detection result corresponding to the standard theoretical calculation result by detecting the test plate, ensuring the authenticity of the detection error and improving the applicability and accuracy of the error detection method.

In some embodiments, the pavement morphology information includes a plurality of elevation values corresponding to each original pavement.

The pavement morphology information may include elevation values at a plurality of different positions corresponding to each original pavement, so as to directly represent the elevation change of the original pavement through the elevation values at different positions.

Referring to FIG. 3 as shown, the pavement morphology information is fitted using a preset data fitting processing algorithm to obtain at least one texture depth variation curve, including:

S210: Dividing the plurality of elevation values into a plurality of first initial elevation value sets based on the regional location information of each of the original pavements, where the plurality of first initial elevation value sets corresponds one-to-one to a plurality of preset geographical areas.

In step S210, the regional location information corresponding to the original pavement is used to characterize the location of the geographical area where the original pavement is located. According to the regional location information of each original pavement, each elevation value in the pavement morphology information can be divided into a plurality of first initial elevation value sets, so that the plurality of first initial elevation value sets corresponds one-to-one to the plurality of preset geographical areas one by one, so as to classify the elevation values in the pavement morphology information according to the different areas where the original pavement is located. The plurality of preset geographical areas can be selected according to the distribution of the original pavement, and the road conditions of the original pavements in the same preset geographical area have certain similarities.

S220: Determining the first initial elevation value set corresponding to the target geographical area as a first target elevation value set, and performing fitting processing on the elevation values in the first target elevation value set to obtain at least one texture depth variation curve.

In step S220, a target geographical area may be selected from the plurality of preset geographical areas, and the target geographical area may be selected according to the actual use area of the pavement wear detection device. After the target geographical area is determined, the first initial elevation value set corresponding to the target geographical area is determined as the first target elevation value set, so as to screen out a first initial elevation value set from the plurality of first initial elevation value sets, and perform fitting processing on the plurality of discrete elevation values in the first target elevation value set, so as to obtain at least one texture depth variation curve corresponding to this part of the elevation values.

The above method is used to generate a texture depth variation curve, so that the texture depth variation curve can match the elevation value corresponding to the target geographical area, so as to characterize the road conditions with certain regularities in the target geographical area through the texture depth variation curve. When determining the detection error later, the detection error of the pavement wear detection device under the road conditions and detection environment with certain geographical area regularities can be more accurately reflected.

In this embodiment, according to the regional location information of each original pavement, a plurality of elevation values are divided into a plurality of first initial elevation value sets, the first initial elevation value set corresponding to the target geographical area is determined as the first target elevation value set, and the elevation values in the first target elevation value set are fitted to obtain at least one texture depth variation curve, thereby achieving the acquisition of the texture depth variation curve, and providing a basis for the generation of the test plate and the determination of the detection error. The regional location information of the original pavement is used as the basis for dividing the elevation values, and the elevation values in the first initial elevation value set corresponding to the target geographical area are fitted, so that the generated texture depth variation curve can match the elevation value corresponding to the target geographical area, ensuring that the final determined detection error can adapt to the road conditions and detection environment with certain geographical regional laws, further improving the authenticity of the detection error and the applicability and accuracy of the error detection method.

In other embodiments, the pavement morphology information includes a plurality of elevation values corresponding to each original pavement respectively.

The pavement morphology information may include elevation values at a plurality of different positions corresponding to each original pavement respectively, so as to directly represent the elevation changes of the original pavement through the elevation values at different positions.

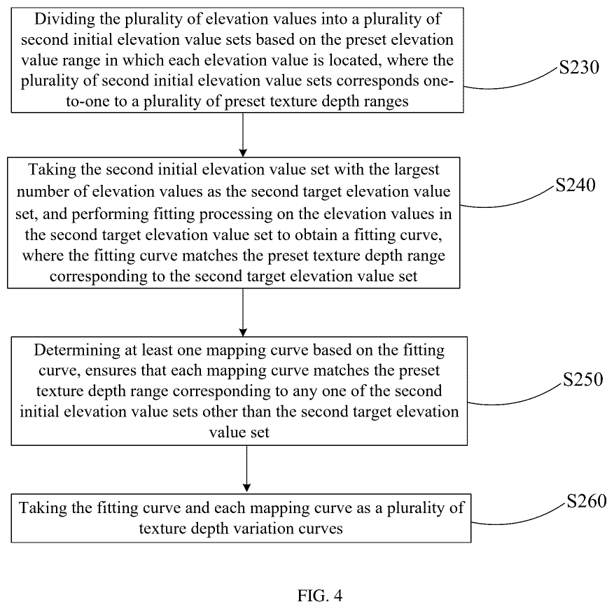

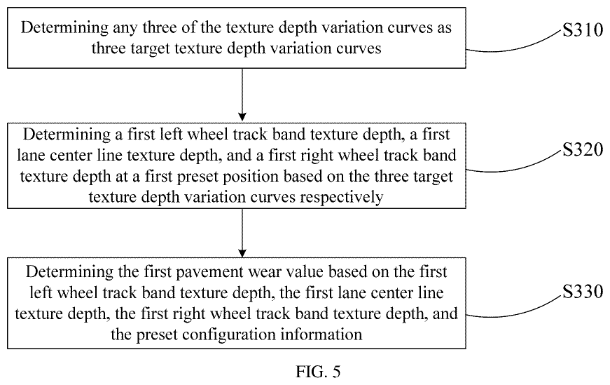

Referring to FIG. 4 as shown, the pavement morphology information is fitted using a preset data fitting processing algorithm to obtain at least one texture depth variation curve, including:

S230: Dividing the plurality of elevation values into a plurality of second initial elevation value sets based on the preset elevation value range in which each elevation value is located, where the plurality of second initial elevation value sets corresponds one-to-one to a plurality of preset texture depth ranges.

In step S230, the plurality of preset elevation value ranges can be determined according to the size of the elevation value, and each elevation value in the pavement morphology information can be divided into a plurality of second initial elevation value sets according to the preset elevation value ranges, so that the plurality of second initial elevation value sets corresponds one-to-one to the plurality of preset texture depth ranges, so as to classify the elevation values in the pavement morphology information according to the different texture depth ranges, and the number and interval size of the preset elevation value ranges can be selected according to the overall distribution of the elevation values.

S240: Taking the second initial elevation value set with the largest number of elevation values as the second target elevation value set, and performing fitting processing on the elevation values in the second target elevation value set to obtain a fitting curve, where the fitting curve matches the preset texture depth range corresponding to the second target elevation value set.

In step S240, after the elevation values are divided into the plurality of second initial elevation value sets, the number of elevation values in each second initial elevation value set is different. The second initial elevation value set with the largest number of elevation values can be used as the second target elevation value set to screen out a second initial elevation value set from the plurality of second initial elevation value sets, and the plurality of discrete elevation values in the second target elevation value set are fitted to obtain a fitting curve corresponding to this part of the elevation values, so that the fitting curve can match the preset texture depth range corresponding to the second target elevation value set.

S250: Determining at least one mapping curve based on the fitting curve, ensures that each mapping curve matches the preset texture depth range corresponding to any one of the second initial elevation value sets other than the second target elevation value set.

In step S250, one or more mapping curves can be generated in a mapping manner based on the obtained fitting curve, so that each mapping curve matches the preset texture depth range corresponding to any other second initial elevation value set, thereby mapping the curve corresponding to the preset texture depth range with the largest amount of elevation value data to other preset texture depth ranges. The mapping curves corresponding to other preset texture depth ranges can be obtained without processing the second initial elevation value set other than the second target elevation value set.

S260: Taking the fitting curve and each mapping curve as a plurality of texture depth variation curves.

In step S260, the fitting curve and the mapping curves are used as a plurality of texture depth variation curves, so that the plurality of texture depth variation curves can match the plurality of preset texture depth ranges, thereby ensuring the diversity of the texture depth variation curves, making the plurality of texture depth variation curves adaptable to a variety of different road conditions, and improving the applicability and accuracy of determining detection errors.

In this embodiment, according to the preset elevation value range in which each elevation value is located, a plurality of elevation values are divided into a plurality of second initial elevation value sets, the second initial elevation value set with the largest number of elevation values is used as the second target elevation value set, and the elevation values in the second target elevation value set are fitted to obtain a fitting curve; at least one mapping curve is determined based on the fitting curve, and the fitting curve and each mapping curve can be used as a plurality of texture depth variation curves, thereby realizing the determination of a plurality of texture depth variation curves, and providing a basis for the generation of the test plate and the determination of the detection error. The preset elevation value range in which the elevation value is located is used as the basis for dividing the elevation values, and only the elevation values in the second initial elevation value set with the largest number of elevation values are fitted, and the mapping curve is determined based on the generated fitting curve, which reduces the amount of calculation while ensuring the authenticity of the mapping curve, and further improves the authenticity of the detection error and the applicability and accuracy of the error detection method.

In some embodiments, the fitting process includes any one or any combination of linear regression fitting processing, polynomial fitting processing, spline interpolation fitting processing, surface fitting processing, and least squares fitting processing.

Any one or any combination of linear regression fitting processing, polynomial fitting processing, spline interpolation fitting processing, surface fitting processing, and least squares fitting processing can be used as a fitting processing method for the plurality of elevation values. When any combination of the above processing is used as a fitting processing method, a preliminary fitting can be performed by one processing method, and another processing method can be selected according to the fitting effect to perform fitting again. It can be understood that the above fitting processing methods can be used when fitting the elevation values in the first target elevation value set or the second target elevation value set.

Wherein, the linear regression fitting process finds a straight line so that the sum of the vertical distances between the straight line and each elevation value in the elevation value set is minimized. It is suitable for elevation values with linear relationships. The calculation is simple and easy to understand and implement. The polynomial fitting process uses a polynomial function to fit the elevation value, which can minimize the sum of the distances between all elevation values and the polynomial function, and can capture elevation values with complex nonlinear relationships. The spline interpolation fitting process fits the elevation value data through piecewise polynomials. Each segment of the polynomial is smoothly connected and can be used to process more complex data. The surface fitting process can be extended to multi-dimensional space. By fitting a multivariate function to describe the distribution of elevation value data, it is suitable for multivariate data fitting processing. The least squares fitting process can determine the function form with the smallest sum of squared errors. It can be applied to scenarios where the fitting effect needs to be optimized and has strong versatility.

In this embodiment, by using any one or any combination of linear regression fitting processing, polynomial fitting processing, spline interpolation fitting processing, surface fitting processing, and least squares fitting processing as a method of fitting the plurality of elevation values, the advantages of each fitting processing method can be utilized to fit a large amount of original data, so that the texture depth variation curve can have a strong fitting effect and meet the needs of different scenarios to characterize complex and diverse road conditions.

In some embodiments, the texture depth includes a left wheel track band texture depth, a lane center line texture depth, and a right wheel track band texture depth, and the number of texture depth variation curves is greater than or equal to 3.

In the correspondence between the texture depth represented by the preset configuration information and the pavement wear value, the texture depth may include the left wheel track band texture depth, the lane center line texture depth, and the right wheel track band texture depth, so that the correspondence represented by the preset configuration information can determine the corresponding pavement wear value according to the left wheel track band texture depth, the lane center line texture depth, and the right wheel track band texture depth. In this case, the standard theoretical calculation principle of the pavement wear value defined by the preset configuration information conforms to the calculation principle of the pavement wear index PWI or the wear rate WR proposed in the “Highway Technical Condition Assessment Standard” (JTG5210-2018), ensuring the accuracy of determining the first pavement wear value and the second pavement wear value based on the preset configuration information.

Exemplarily, when the texture depth includes the left wheel track band texture depth MPD_{L}, lane center line texture depth MPD_{C} and right wheel track depth MPD_{R}, the wear rate WR and the pavement wear index PWI are determined by the following formulas (1) and (2), respectively:

$$WR = 100 \times \frac{{MPD}_{C} - min \{ {MPD}_{L} , {MPD}_{R} \}}{{MPD}_{C}} \qquad ( 1 )$$

$$PWI = 100 - {a}_{0} {WR}^{{a}_{1}} \qquad ( 2 )$$

wherein, a₀ and a₁ are model parameters, exemplarily, a₀ can be 1.696, and a₁ can be 0.785.

In this case, when determining the first pavement wear value and the second pavement wear value, in order to simultaneously provide the left wheel track band texture depth, the lane center line texture depth and the right wheel track band texture depth, it is necessary to ensure that the number of texture depth variation curves is greater than or equal to 3, so that the number of test plates is also greater than or equal to 3, so as to provide the above three texture depths when determining the first pavement wear value through at least three texture depth variation curves, and provide the above three texture depths when determining the second pavement wear value through at least three test plates. When the number of texture depth variation curves is greater than three, the first pavement wear value and the second pavement wear value corresponding to a plurality of different situations can be determined based on the arrangement and combination of any three texture depth variation curves and their corresponding test plates.

In this embodiment, the left wheel track band texture depth, the lane center line texture depth and the right wheel track band texture depth are used as texture depths at the same time, which provides a theoretical basis and calculation method that meets the standard for determining the pavement wear value, and ensures the accuracy of determining the first pavement wear value and the second pavement wear value based on the preset configuration information. The number of texture depth variation curves is set to be greater than or equal to 3, so that when determining the first pavement wear value and the second pavement wear value, the left wheel track band texture depth, the lane center line texture depth and the right wheel track band texture depth can be provided at the same time, and when the number of texture depth variation curves is greater than 3, the corresponding first pavement wear value and the second pavement wear value under a plurality of different conditions can be obtained through the arrangement and combination of any three texture depth variation curves and their corresponding test plates, which increases the data volume of the first pavement wear value and the corresponding second pavement wear value, and further improves the accuracy of the detection error.

In some embodiments, referring to FIG. 5 as shown, determining the first pavement wear value based on each texture depth variation curve and the preset configuration information, including:

S310: Determining any three of the texture depth variation curves as three target texture depth variation curves.

In step S310, when the texture depth includes the left wheel track band texture depth, the lane center line texture depth, and the right wheel track band texture depth, and the first pavement wear value according to each texture depth variation curve and the preset configuration information is determined, it is necessary to simultaneously provide three texture depths as the left wheel track band texture depth, the lane center line texture depth, and the right wheel track band texture depth through the texture depth variation curve, so as to provide a basis for determining the first pavement wear value. At this point, any three texture depth variation curves can be selected from at least three texture depth variation curves as three target texture depth variation curves.

S320: Determining a first left wheel track band texture depth, a first lane center line texture depth, and a first right wheel track band texture depth at a first preset position based on the three target texture depth variation curves respectively.

In step S320, after determining the three target texture depth variation curves, the texture depths corresponding to the three target texture depth variation curves at the first preset position are determined respectively according to the three target texture depth variation curves, so as to serve as the first left wheel track band texture depth, the first lane center line texture depth and the first right wheel track band texture depth respectively. The first preset position can be selected according to the length of the three target texture depth variation curves and the detection accuracy of the pavement wear detection device, so as to ensure that the three target texture depth variation curves have texture depths capable of determining the pavement wear value at the first preset position, and to ensure that the pavement wear detection device can realize the actual detection of the pavement wear value at the corresponding position of the test plate.

S330: Determining the first pavement wear value based on the first left wheel track band texture depth, the first lane center line texture depth, the first right wheel track band texture depth, and the preset configuration information.

In step S330, since the preset configuration information can characterize the correspondence between the left wheel track band texture depth, the lane center line texture depth and the right wheel track band texture depth and the pavement wear value when the texture depth includes the left wheel track band texture depth, the lane center line texture depth and the right wheel track band texture depth, the first pavement wear value can be determined according to the first left wheel track band texture depth, the first lane center line texture depth, the first right wheel track band texture depth and the preset configuration information, so that the first pavement wear value is the pavement wear value under the standard theoretical calculation condition corresponding to the target texture depth variation curve, i.e., the numerical curve, which represents the detection result that should be obtained if the pavement wear detection device has no detection error, and provides a basis for comparison with subsequent actual measurement results.

The detecting the test plate with the pavement wear detection device, so that the second pavement wear value is determined based on the test plate and the preset configuration information with the pavement wear detection device includes: detecting the three test plates generated respectively based on the three target texture depth variation curves with the pavement wear detection device, so that the second left wheel track band texture depth, the second lane center line texture depth and the second right wheel track band texture depth at the second preset position are determined based on the three test plates respectively with the pavement wear detection device, and the second pavement wear value is determined based on the second left wheel track band texture depth, the second lane center line texture depth, the second right wheel track band texture depth and the preset configuration information; wherein the first preset position of the target texture depth variation curve matches the second preset position of the test plate.

In the case where the texture depth includes the left wheel track band texture depth, the lane center line texture depth and the right wheel track band texture depth, when the test plate is detected with the pavement wear detection device, so that the pavement wear detection device determines the second pavement wear value based on the test plate and preset configuration information, it is necessary to simultaneously provide three texture depths through the test plate as the left wheel track band texture depth, the lane center line texture depth and the right wheel track band texture depth for providing a basis for determining the second pavement wear value.

At this point, the pavement wear detection device can be used to detect the three test plates generated respectively based on the three target texture depth variation curves. The pavement wear detection device can respectively determine the texture depths corresponding to the three test plates at the second preset positions according to the three test plates, as the second left wheel track band texture depth, the second lane center line texture depth and the second right wheel track band texture depth.

Since the preset configuration information can characterize the correspondence between the left wheel track band texture depth, the lane center line texture depth and the right wheel track band texture depth and the pavement wear value when the texture depth includes the left wheel track band texture depth, the lane center line texture depth and the right wheel track band texture depth; the second pavement wear value can be determined according to the second left wheel track band texture depth, the second lane center line texture depth, the second right wheel track band texture depth and the preset configuration information, so that the second pavement wear value is the pavement wear value when the test plate is actually tested by the pavement wear detection device using the same calculation theory, reflecting the detection effect of the pavement wear detection device on the real pavement.

The first preset position of the target texture depth variation curve matches the second preset position of the test plate, making the first pavement wear value and the second pavement wear value are both based on the same preset configuration information, that is, the pavement wear value at the same position determined by the calculation theory, thereby ensuring that the difference between the first pavement wear value and the second pavement wear value can represent the difference between the standard theoretical calculation result and the actual detection result of the pavement wear detection device, so as to realize the determination of the detection error through the first pavement wear value and the second pavement wear value.

In this embodiment, any three texture depth variation curves are determined as three target texture depth variation curves, and the first left wheel track band texture depth, the first lane center line texture depth and the first right wheel track band texture depth of the first preset position are determined respectively according to the three target texture depth variation curves. According to the first left wheel track band texture depth, the first lane center line texture depth, the first right wheel track band texture depth and preset configuration information, the first pavement wear value can be determined, which provides a basis for comparison with the subsequent second pavement wear value. The pavement wear detection device is used to detect three test plates generated based on three target texture depth variation curves, so that the pavement wear detection device can determine the second left wheel track band texture depth, the second lane center line texture depth and the second right wheel track band texture depth at the second preset position based on the three test plates respectively, and can determine the second pavement wear value according to the second left wheel track band texture depth, the second lane center line texture depth, the second right wheel track band texture depth and the preset configuration information, ensuring that the difference between the first pavement wear value and the second pavement wear value can represent the difference between the standard theoretical calculation result and the actual detection result of the pavement wear detection device at the same position, providing a basis for determining the detection error.

In some embodiments, different target texture depth variation curves are matched with different preset texture depth ranges. Among the maximum values of the three preset texture depth ranges matched by the three target texture depth variation curves, the maximum value of the preset texture depth range matched with the target texture depth variation curve used to determine the first lane center line texture depth is the smallest.

Different target texture depth variation curves are configured to match different preset texture depth ranges, and the maximum value of the preset texture depth range matched with the target texture depth variation curve for determining the first lane center line texture depth is the smallest among the maximum values of the three preset texture depth ranges matched by the three target texture depth variation curves. It is possible to ensure that the first left wheel track band texture depth, the first lane center line texture depth and the first right wheel track band texture depth determined according to the three target texture depth variation curves can be in different preset texture depth ranges, so as to restore the real and complex road conditions through the difference and diversity of the numerical ranges between the first left wheel track band texture depth, the first lane center line texture depth and the first right wheel track band texture depth, and ensure the accuracy and reference value of the calculation result of the first pavement wear value. And ensure the first lane center line texture depth is smaller than the first left wheel track band texture depth and the first right wheel track band texture depth, so as to restore the normal wear of the pavement under driving conditions.

As mentioned above, when generating a texture depth variation curve, a fitting curve can be obtained by fitting the elevation values in the second target elevation value set, and at least one mapping curve can be obtained by mapping according to the fitting curve, so that the fitting curve and each mapping curve match different preset texture depth ranges. In this case, when three of the plurality of texture depth variation curves are selected as target texture depth variation curves, the three target depth texture depth variation curves can be matched with three different preset texture depth ranges, and the target texture depth variation curve that matches the preset texture depth range with the smallest maximum value is selected as the target texture depth variation curve for determining the first lane center line texture depth.

In this embodiment, different target texture depth variation curves are configured to match different preset texture depth ranges, and among the three preset texture depth ranges respectively matched by the three target texture depth variation curves, the maximum value of the preset texture depth range matched with the target texture depth variation curve used to determine the first lane center line texture depth is the smallest, which can restore the real and complex road conditions and the normal wear of the pavement under driving conditions, and further improve the accuracy and reference value of determining the first pavement wear value based on the first left wheel track band texture depth, the first lane center line texture depth and the first right wheel track band texture depth.

After generating the test plates corresponding to each texture depth variation curve based on each texture depth variation curve, the error detection method of the pavement wear detection device further includes: performing process error detection on each generated test plate to determine the dimensional error between the extension path of the edge of the test plate and the texture depth variation curve caused by the processing technology of the test plate.

It is understandable that, due to the limitation of the processing technology when the test plate is generated, there may be certain dimensional errors between the extension path of the edge of the test plate generated according to each texture depth variation curve and the corresponding texture depth variation curve. After the test plate is generated, the generated test plates can be subjected to process error detection to determine the dimensional error of each test plate through process error detection. Exemplarily, the test plate can be subjected to process error detection by a probe of a high-precision three-dimensional coordinate measuring machine to determine the dimensional error between each test plate and the corresponding texture depth variation curve.

The detecting the test plate with the pavement wear detection device includes: detecting the test plate whose size error is less than the preset error with the pavement wear testing device.

When the size error is too large, it represents a significant difference between the extension path of the edge of the test plate and the corresponding texture depth variation curve, and the test plate does not restore the road conditions represented by the texture depth variation curve to an insufficient degree, making the difference between the first pavement wear value and the second pavement wear value also includes the difference between the test plate and the texture depth variation curve, and the detection error of the pavement wear detection device cannot be accurately determined by the first pavement wear value and the second pavement wear value. Therefore, when detecting the test plate with the pavement wear detection device, detecting the test plate whose size error is less than the preset error, so as to control the size error of the test plate actually used for detection within the preset error, so that the second pavement wear value can correspond to the actual detection result under the same road conditions as the first pavement wear value, and ensure the reference value provided by the second pavement wear value for determining the detection error.

In this embodiment, by performing process error detection on each generated test plate to determine the dimensional error between the extension path of the edge of the test plate and the texture depth variation curve caused by the processing technology of the test plate, the pavement wear detection device can be used to detect the test plate whose dimensional error is less than the preset error, thereby achieving control over the dimensional error of the test plate caused by the manufacturing process, and ensuring that the second pavement wear value obtained by the pavement wear detection device when detecting the test plate can correspond to the first pavement wear value determined according to the texture depth variation curve, thereby further improving the accuracy of determining the detection error based on the first pavement wear value and the second pavement wear value.

In some embodiments, the test plate is generated by any one or a combination of three-dimensional printing, precision machining, and three-axis computer numerical control machining.

Any one or any combination of three-dimensional printing, precision machining, and three-axis computer numerical control machining can be used as a method for generating a test plate, so as to generate test plates that correspond one-to-one with each texture depth variation curve according to each texture depth variation curve, and ensure that the extension path of the edge of the test plate is consistent with the corresponding texture depth variation curve.

Wherein, 3D printing creates 3D objects by stacking materials layer by layer, which can realize the rapid and batch generation of test plates based on 3D data, and is suitable for making complex plate structures. Precision machining uses high-precision mechanical or electronic means to cut, grind and other operations on materials to obtain high-precision and high-quality test plates, which is suitable for making structures with strict dimensional requirements. Three-axis computer numerical control machining, or three-axis CNC machining, uses tools that move along three axes to process fixed workpieces, and has the characteristics of low cost and simple operation.

In this embodiment, any one or any combination of three-dimensional printing, precision machining, and three-axis computer numerical control machining is used as a method for generating the test plate, which can achieve the generation of test plates that correspond one-to-one to each texture depth variation curve, and provide a basis for determining the second pavement wear value. The appropriate generation method can be selected according to the needs to ensure the production accuracy of the test plate and control the production cost of the test plate.

In some embodiments, the material of the test plate is aluminum, the thickness of the test plate is 0.5-1.5 cm, and the surface of the test plate is matte treated.

Setting the material of the test plate to aluminum enables it to have characteristics such as lightweight, high strength, corrosion resistance, easy processing, recyclability, and high fatigue strength. This makes the test plate easy to produce and arrange, reusable and can carry pavement wear detection device. The thickness of the test plate is set to 0.5-1.5 cm, and the surface of the test plate is matte treated, so that the test plate can have the same texture depth variation as the real pavement, adapt to the detection accuracy of the pavement wear detection device, and improve the degree of restoration of the test plate to the real pavement.

In this embodiment, the material of the test plate is set to aluminum, the thickness is set to 0.5-1.5 cm, and the surface of the test plate is matte treated, so that the test plate is easy to produce and arrange, reusable and has a certain bearing capacity, which improves the degree of restoration of the test plate to the real pavement, and is conducive to using the test plate as a detection object of the pavement wear detection device to realize the determination of the second pavement wear value.

In some embodiments, the error detection method of the pavement wear detection device further includes: based on the detection error, performing any one or any combination of the following processing on the pavement wear detection device: detection error calibration processing, detection error cause analysis processing, and detection parameter correction processing.

After determining the detection error of the pavement wear detection device based on the first pavement wear value and the second pavement wear value, the pavement wear detection device can be subjected to at least one of detection error calibration processing, detection error cause analysis processing and detection parameter correction processing based on the detection error, so as to realize calibration, analysis or correction of the detection error of the pavement wear detection device.

Wherein, the detection error calibration processing is used to calibrate the current detection error of the pavement wear detection device, and present the detection error that will exist if the pavement wear detection device continues to be put into use in a numerical form, so that the user can choose whether to continue to use the pavement wear detection device based on the calibrated detection error, or adjust the detection result based on the calibrated detection error, enables the adjusted detection result is closer to the actual result.

The analysis and processing of the cause of detection error is used to analyze the cause of the detection error of the pavement wear detection device based on the detection error. Exemplarily, a plurality of detection errors can be determined multiple times through the above method. If the difference between the plurality of detection errors is small, it can be considered that the pavement wear detection device has a detection error due to its own reasons, such as equipment aging, component failure, etc., so as to repair or maintain the pavement wear detection device. If the difference between the plurality of detection errors is large, it can be considered that the pavement wear detection device has a detection error due to complex and changeable road conditions or external environmental factors, and a pavement wear detection device that is more suitable for the current road conditions or environment can be selected for detection.

The detection parameter correction processing is used to debug the equipment parameters of the pavement wear detection device, so as to perform appropriate correction on the detection parameters through the feedback of the detection errors, thereby eliminating or reducing the detection errors of the pavement wear detection device after the parameter correction. Exemplarily, the model parameters in the preset configuration information adopted by the pavement wear detection device can be adjusted according to the detection error to change the corresponding relationship between the texture depth and the pavement wear value, making the final output the pavement wear value more accurate.

In this embodiment, based on the detection error, the pavement wear detection device is subjected to calibration for detection error, analysis processing of detection error causes, and correction processing of detection parameters. This enables calibration, analysis or correction of the detection error of the pavement wear detection device, which is conducive to selecting suitable pavement wear detection device for different scenarios, and can ensure the stability and accuracy of the detection results of the pavement wear detection device, thereby improving the user experience of the pavement wear detection device.

Those skilled in the art will readily appreciate other embodiments of the present disclosure after considering the specification and practicing the invention disclosed herein. The present application is intended to cover any variations, uses or adaptations of the present disclosure that follow the general principles of the present disclosure and include common knowledge or customary techniques in the art that are not disclosed in the present application. The specification and examples are to be considered as exemplary only, and the true scope and spirit of the present disclosure are indicated by the following claims.

It should be understood that the present disclosure is not limited to the exact construction that has been described above and shown in the drawings and that various modifications and changes may be made without departing from the scope thereof. The scope of the present disclosure is limited only by the appended claims.

1. An error detection method of a pavement wear detection device, comprising:
acquiring pavement morphology information, wherein the pavement morphology information is used to characterize elevation changes of a plurality of original pavements;
performing fitting processing on the pavement morphology information by using a preset data fitting processing algorithm to obtain at least one texture depth variation curve;
determining a first pavement wear value based on each texture depth variation curve and preset configuration information, wherein the preset configuration information is used to characterize a corresponding relationship between a texture depth and a pavement wear value;
generating a test plate in one-to-one correspondence with each texture depth variation curve based on each texture depth variation curve, wherein an extension path of an edge of the test plate is consistent with the corresponding texture depth variation curve;
detecting the test plate with the pavement wear detection device, so that a second pavement wear value is determined based on the test plate and the preset configuration information with the pavement wear detection device; and
determining a detection error of the pavement wear detection device based on the first pavement wear value and the second pavement wear value;
wherein after generating the test plate in one-to-one correspondence with each texture depth variation curve based on each texture depth variation curve, the error detection method of the pavement wear detection device further comprises:
performing a process error detection on each generated test plate to determine a dimensional error between the extension path of the edge of the test plate and the texture depth variation curve caused by processing technology of the test plate;
wherein the detecting the test plate with the pavement wear detection device comprises:
detecting the test plate whose dimensional error is less than a preset error with the pavement wear detection device.

2. The error detection method of the pavement wear detection device according to claim 1, wherein the pavement morphology information includes a plurality of elevation values corresponding to each of the original pavements respectively;
the performing fitting processing on the pavement morphology information by using a preset data fitting processing algorithm to obtain at least one texture depth variation curve includes:
dividing the plurality of elevation values into a plurality of first initial elevation value sets based on regional location information of each of the original pavements, wherein the plurality of first initial elevation value sets corresponds one-to-one to a plurality of preset geographical areas;
determining the first initial elevation value set corresponding to the target geographical area as a first target elevation value set, and performing fitting processing on the elevation values in the first target elevation value set to obtain at least one texture depth variation curve; or,
dividing the plurality of elevation values into a plurality of second initial elevation value sets based on the preset elevation value range in which each elevation value is located, wherein the plurality of second initial elevation value sets corresponds one-to-one to a plurality of preset texture depth ranges;
taking the second initial elevation value set having a largest number of elevation values as a second target elevation value set, and performing fitting processing on the elevation values in the second target elevation value set to obtain a fitting curve, wherein the fitting curve matches the preset texture depth range corresponding to the second target elevation value set;
determining at least one mapping curve based on the fitting curve, ensuring that each mapping curve matches the preset texture depth range corresponding to any one of the second initial elevation value sets other than the second target elevation value set; and
taking the fitting curve and each mapping curve as a plurality of texture depth variation curves.

3. The error detection method of the pavement wear detection device according to claim 2, wherein the fitting processing comprises any one or any combination of linear regression fitting processing, polynomial fitting processing, spline interpolation fitting processing, surface fitting processing, and least squares fitting processing.

4. The error detection method of the pavement wear detection device according to claim 1, wherein the texture depth comprises a left wheel track band texture depth, a lane center line texture depth and a right wheel track band texture depth;
a number of the texture depth variation curve is greater than or equal to 3.

5. The error detection method of the pavement wear detection device according to claim 4, wherein the determining a first pavement wear value based on each texture depth variation curve and preset configuration information comprises:
determining any three of the texture depth variation curves as three target texture depth variation curves;
determining a first left wheel track band texture depth, a first lane center line texture depth and a first right wheel track band texture depth at a first preset position based on the three target texture depth variation curves respectively; and
determining the first pavement wear value based on the first left wheel track band texture depth, the first lane center line texture depth, the first right wheel track band texture depth and the preset configuration information;
wherein the detecting the test plate with the pavement wear detection device, so that a second pavement wear value is determined based on the test plate and the preset configuration information with the pavement wear detection device comprises:
detecting three test plates generated respectively based on the three target texture depth variation curves with the pavement wear detection device, ensuring that a second left wheel track band texture depth, a second lane center line texture depth and a second right wheel track band texture depth at a second preset position are determined based on the three test plates respectively with the pavement wear detection device, and the second pavement wear value is determined based on the second left wheel track band texture depth, the second lane center line texture depth, the second right wheel track band texture depth and the preset configuration information; wherein the first preset position of the target texture depth variation curve matches the second preset position of the test plate.

6. The error detection method of the pavement wear detection device according to claim 5, wherein different target texture depth variation curves match different preset texture depth ranges;
among maximum values of three preset texture depth ranges respectively matched by the three target texture depth variation curves, a maximum value of the preset texture depth range matched with the target texture depth variation curve used to determine the first lane center line texture depth is a smallest.

7. The error detection method of the pavement wear detection device according to claim 1, wherein the generation method of the test plate comprises any one or a combination of three-dimensional printing, precision machining, and three-axis computer numerical control machining.

8. The error detection method of the pavement wear detection device according to claim 1, wherein a material of the test plate is aluminum, a thickness of the test plate is 0.5-1.5 cm, and a surface of the test plate is matte treated.

9. The error detection method of the pavement wear detection device according to claim 1, wherein the error detection method of the pavement wear detection device further comprises:
based on the detection error, performing any one or any combination of the following processes to the pavement wear detection device:
detection error calibration processing;
analysis and processing of the cause of detection error;
detection parameter correction processing.
